(19) United States
(12) Patent Application Publication
(10) Pub. No.: US 2007/0027414 A1
(43) Pub. Date: Feb. 1, 2007

(54) Laminar construction negative pressure wound dressing including bioabsorbable material

(75) Inventors: Brian D. Hoffman, Princeton, NJ (US); Robert A. Rabiner, North Reading, MA (US)

Correspondence Address: FULWIDER PATTON, 6060 CENTER DRIVE, 10TH FLOOR, LOS ANGELES, CA, 90045, US

(73) Assignee: Integra LifeSciences Corporation

(21) Appl. No.: 11/193,651

(22) Filed: Jul. 28, 2005

(51) Int. Cl. A61F 5/00

(52) U.S. Cl. 602/2

(57) ABSTRACT

A laminated negative pressure wound dressing system and method is described. The wound dressing is disposed in the wound in layers including at least one bioabsorbable layer that contacts the wound bed, a bioabsorbable fluid communicating layer, an atmospheric barrier layer, and a tube for applying a negative pressure to the wound bed. Ingrowth of granulation tissue into the bioabsorbable wound bed layer does not need to be inhibited as the bioabsorbable material need not be removed during dressing changes. A kit containing the components of the wound dressing system is also disclosed as well as a method for applying the dressing.

### FIELD OF THE INVENTION

The present invention relates generally to wound care and more particularly to negative pressure wound dressings and negative pressure dressing kits.

### BACKGROUND OF THE INVENTION

It is well known to medical science that wounds under greater than atmospheric pressure do not heal well. When a wound is under high pressure, infection tends to spread and blood circulation to the wound bed is impaired. Also, such elevated pressure in the wound prevents healthy tissue growth and impairs the formation of granulation tissue. In fact, incision and drainage of infected wounds dates back to early recorded history. By incising an abscess, the wound drains and the pressures in the wound reach a level of equilibrium with an atmospheric level of pressure. Furthermore, wound healing is facilitated by removing excess air, fluid, and debris, the presence of which usually inhibits the normal healing process.

Although the preferable method of treating a clean wound is by primary closure with sutures or staples, sometimes closure by such techniques is impossible. For example, sometimes the amount of tissue loss in a wound does not allow approximation of the wound edges without undue mechanical stress. Such mechanical stresses on sutures must be avoided because of risk of wound dehiscence. Other times, wounds cannot be closed primarily because of the presence of infection or the hardening of wound edges by scar tissue and inflammation. Yet other types of wounds that are not amenable to primary closure are decubitus ulcers, large deep wounds, infected wounds, and shallow wide wounds where skin loss prevents wound closure without skin grafting.

Chronic wounds, such as pressure wounds may take months or years to heal without primary closure. Long healing times often reduce patient mobility, thereby resulting in additional medical complications and further exacerbating the patient's underlying medical condition. Additional decubitus ulcers may occur during immobilization, as well as more serious complications, for example, thrombophlebitis and pulmonary embolism.

Chronic wound management starts with proper cleaning and debriding of the wound and use of sterile wound dressing changes. Frequent wound dressing changes are needed to remove the fluids produced by the wound, many of which inhibit wound healing. Wound exudates can result in bacterial colonization and often lead to inflammation of the wound and a delayed healing response.

For many centuries, drains have been placed within open and closed wounds or the body cavity to aid in the healing process. Drains may take the form of a simple ribbon like strip of material such as iodoform gauze, 4×4 gauze packing, or collapsible rubber tubes such as the popular “Penrose” drain. Placement of a drain within a wound establishes a path for drainage of fluids and blood, cellular debris, and infected exudates out of the body. The drain keeps the superficial parts of the wound from closing off before the deeper parts of the wound have completely granulated in and filled the wound defect. If the skin and superficial part of a wound close before the deeper layers have healed, pressure will again build up in the wound resulting in delayed wound healing or an infection. A drain prevents pressure from building up in the wound by allowing the body to fully granulate in the depths of the wound before superficial epithelialization of the wound is allowed. The drain also acts as a pressure release conduit from the depths of the wound to the surface.

Suction drainage, an improvement over the simple passive drains discussed above, has been used in medicine since 1947. These types of drains are commonly placed at the time of a surgical procedure where postoperative accumulation of blood, bile, or exudates is expected. Suction drains may be connected to a wall or electrical vacuum pump. More typically, however, these drains are connected to a portable canister. Typical kinds of portable suction drains used in U.S. hospitals today include the Jackson-Pratt drain and the Hemovac Drain. Both of these drains have self contained portable canisters. The Jackson-Pratt drain is usually supplied with a canister that is grenade shaped, and made of soft plastic or silicone. The distal end of the Jackson-Pratt drain is inserted into a wound and is a semi-rigid, rubber or silicone, round or flat, generally tubular structure with a central channel that is in fluid communication with multiple perforations to the exterior of the drain. The wound is then usually sutured closed over the distal end of the drain. Tubing connects the central channel of the drain to the grenade. The grenade is compressed, connected to the tubing, and then sealed, thus exerting suction on the distal end and creating negative pressure within the wound as the grenade tends to return to its expanded state. The Hemovac drain is a similar device, but has a canister with a spring that encourages the canister to expand, thus providing the suction force on the distal end of the drain within the wound. The Hemovac drain usually includes a single tube that connects with the canister on one end, and has multiple perforations from a central channel to the exterior of the drain on the other, distal end. The distal end of both types of drains can be trimmed to length by the clinician. Both devices require a sealed wound to function properly. Otherwise the canisters fully expand and the suction effect is lost. The canister can also be connected to wall suction for even greater and more constant suction forces. On occasion, these drains can be used in combination with an irrigation system that slowly drips saline irrigation into a wound while the drain sucks the fluid out. In suction irrigation systems, there is still a negative pressure environment maintained in the wound since suction forces predominate over the inflow from the irrigation catheters.

It has more recently been recognized that placing a wound under negative pressure speeds up the healing process. Vacuum drainage is felt to encourage wound healing by reducing bacterial counts and by increasing blood flow up to four times above baseline levels. Negative pressure wound therapy is felt to work by minimizing interstitial edema, decompressing small vessels and encouraging local blood flow, and removing wound fluids containing matrix metalloproteinase (MMPs) which can inhibit wound healing. Other authors have felt that proliferation of fibroblasts, endothelial cells, and vascular smooth muscle is encouraged by mechanically deforming these cells. Negative pressures of up to 150 mm Hg have generally found to be beneficial, while negative pressures exceeding 400 mm Hg are generally detrimental and inhibit blood flow.

Dr. Mark Chariker described a basic method for negative pressure wound therapy in “Effective management of incisional and cutaneous fistulae with closed suction wound drainage”, Contemporary Surgery, June 1989. The technique is described for ventral enterocutaneous fistula but is equally applicable to other wounds. The Chariker system was devised with the intention of collecting drainage, obviating skin damage, improving wound granulation and contraction, and minimizing dressing changes. Dr. Chariker emphasizes that a dressing that conforms to the wound bed, combined with continuous closed suction that removes effluent from the wound and creates a lowered pressure in the wound, is critical to the success of his system. He further noted that an occlusive dressing maintains adequate hydration of the tissue and prevents eschar formation. The system decreases inflammatory response, thereby increasing the rate of re-epithelialization.

Dr. Chariker described a kit that contains components that are readily available at any hospital. The kit includes one Jackson Pratt drain, two-by-two inch (2×2) and four-by-four inch (4×4) gauze pads, normal saline, a “Christmas tree” adapter, skin sealant, transparent adhesive film dressing to seal the wound site, Stomahesive® Paste, tape, and a continuous suction system.

The Chariker closed wound drainage method involves irrigating the wound with normal saline, placing the Jackson Pratt drain in the wound bed, packing the wound and covering the drain with saline-saturated four-by-four gauze pads, applying skin sealant to the skin, cutting the transparent film dressing to cover at least one inch of skin beyond wound edges, placing the film dressing over the packed wound and splitting the film dressing to wrap around the Jackson Pratt tubing, placing Stomahesive® Paste to form an airtight seal where the tube exits the film dressing and reinforcing the seal with waterproof pink tape, connecting the Jackson Pratt tube to a continuous suction system using a “Christmas tree” adapter, and turning on a continuous suction in the range of 60-80 mm Hg. With the Chariker system, not only is the wound drainage removed, but the sealing of the wound through the gauze pads, tape, and paste results in the wound being under constant negative pressure. Chariker states that the dressing should be changed every 72 to 120 hours depending on the dressing type and the amount of drainage.

The main advantage of the Chariker system is that it is inexpensive and uses readily available ordinary hospital supplies. However, the Chariker system does have several disadvantages. Because applying this type of negative pressure dressing is technically challenging, the staff must be well educated and experienced. The failure of negative pressure wound therapy is often due to inadequate staff education and skill. Good results are highly dependent on the clinician's technique, as applying presently available negative pressure dressing materials is complicated and awkward. If the packing does not properly conform to the wound or negative pressure is not maintained under the film dressing, the system fails, according to Chariker.

In the Chariker system, the Jackson Pratt drain must be exactly placed within the wound for the system to work. The film dressing must provide a perfect seal around the drain and be attached securely to the skin without causing unnecessary skin irritation. The supplies must be assembled by the hospital staff. The clinician must determine in advance how many 4×4 or 2×2 gauze pads will be needed and pre-soak these in saline. Often the saline soaked gauze pads will wet the patient's bedding or gown, resulting in additional staff time and effort to clean up after a dressing change.

Furthermore, the Chariker system carries risks of severe complications when used with very large wounds. Large wounds require using a large number of gauze pads. It is possible to miss seeing and feeling a gauze pad deep in a wound and thus neglect to remove all of the old gauze when doing dressing changes. Therefore, gauze pads must be carefully counted during placement to assure that no old gauze pads are left in the wound during a subsequent dressing change. Unintentionally leaving a gauze pad deep in a wound for a prolonged period of time could be disastrous with a resultant severe foreign body reaction and almost certain infection.

The use of gauze pads to fill the wound has other disadvantages. Gauze pads are not uniformly porous; therefore they will not distribute the suction forces from the Jackson Pratt drain in a uniform manner. A uniform negative pressure may be very difficult to achieve throughout the wound cavity, and there may not be negative pressures at all in some corners and recesses of the wound. The gauze pad fibers may enter the perforations in the Jackson Pratt drain resulting in occlusion of sections of the drain. The Jackson Pratt drain central channel may also get clogged with blood clot or debris, since the gauze may not provide a consistent barrier to entry of these materials into the drain. Furthermore, granulation tissue growing from the wound may infiltrate the gauze. When the gauze is removed, there may be pain and bleeding.

Chariker emphasizes that the gauze must precisely conform to the wound bed to be effective. It is difficult to tell how much saline soaked gauze to place in a wound. Once the would, drain, and gauze pad are covered with transparent dressing and suction applied, there may be too little gauze to properly fill the wound. Alternatively, too much gauze will mechanically force the wound edges open and slow wound healing. It is very difficult to estimate the proper amount of wet gauze at a dressing change. The volume of the saline soaked gauze will change significantly as soon as suction force is applied and the saline is withdrawn from the wound. If there is not enough gauze, the dressing will need to be redone. The transparent film dressing will need to be removed, more gauze added, and a new transparent film dressing applied. These errors can occur frequently when an inexperienced clinician applies the vacuum dressing. Having to redo a dressing is not only expensive in terms of time and supplies, but is also a very inefficient use of a limited nursing staff.

Another wound treatment employing reduced pressure is disclosed in U.S. Pat. No. 5,636,643 to Argenta et al. Argenta discloses a fluid or gas impermeable wound cover, such as an Ioban adhesive sheet, sealed over a wound site filled with an open cell polyester foam or polyurethane foam, whereby a vacuum pump supplies suction within the wound cover over the treatment site through a tube imbedded in the foam. Argenta also describes a reduced pressure appliance made from a CPR mask and a screen formed of a perforated polymer surgical mesh, such as Prolene mesh, or alternatively a section of honeycombed polyethylene sheet. As disclosed by Argenta, the sealing means for a pressure appliance may include a separate sealing member such as an adhesive strip or a sealing ring. Argenta describes the porous wound screen in the form of a sponge or open cell foam material for placement in the wound. However, none of the materials disclosed by Argenta are bioabsorbable materials.

Still another negative pressure wound therapy device is disclosed in U.S. Pat. No. 6,695,823 to Lina et al. Lina discloses a vacuum pump, and a porous wound pad that is placed over or within a wound and adhesively secured thereto. Lina states that the pad contains multiple pore sizes to prevent granulation tissue from migrating into the pad. Lina further states that the pad has an outer surface adjacent the wound with pore sizes of a diameter of approximately one-hundred microns or less to prevent tissue from growing into the pad. Lina uses a smaller pore size adjacent the wound bed to try to solve the problem of ingrowth of tissue into the pad. Lina states that an objective is to have a pad that (a) is made from biocompatible material and (b) has sufficiently small pore size that granulation tissue does not migrate into the pad. Lina therefore also teaches the avoidance of cell growth into the pad due to the possibility of pain and bleeding when the pad is later removed from the wound, as was discussed above. Lina also attempts to solve the problem of growth of granulation tissue into the pad by altering the outer pore size of the pad or coating the pad with various growth inhibiting chemicals, such as antimicrobial agents.

The disadvantage of the Lina pad is that although it is biocompatible, it is not bioabsorbable. This is a concern because if the Lina pad is cut to a smaller size, small pieces or dust-like particles of the pad material will inevitably adhere to the pad or possibly fall into the wound during the cutting process. These small particles will ultimately contaminate the wound and cause foreign body reactions. These small non-absorbable particles will be a nidus for infection in wounds that no doubt already have a significant bacterial count.

Furthermore, sometimes multiple pieces of pad are used together in certain wounds. Leaving behind in a wound one piece of non-absorbable pad during dressing changes is an inherent risk of using non-absorbable materials and could be disastrous. A piece of non-absorbable pad inadvertently left in a wound for weeks will result in the wound not healing and probably becoming infected. The experienced clinician therefore will count and record the number of pieces of pad inserted into the patient's wound to assure that all pieces are retrieved at the subsequent dressing change. This is time consuming and not fool proof as often more than one clinician is doing the dressing changes.

In addition, it is cumbersome for a clinician to be required to determine which side of the pad has the small pores, and therefore is the wound side, and which part of the pad has the larger pores that cannot be placed against the wound without risking ingrowth of granulation tissue. It is also impractical to cut such a pad into small pieces to conform to the wound bed while at the same time being mindful of not placing the part of a pad with larger pores against the wound.

Modern negative pressure wound dressings are manufactured by companies such as Blue Sky Medical, Inc. and Kinetic Concepts, Inc. These dressing kits typically contain a sheet of transparent adhesive film, a pad of non-bioabsorbable open cell foam (porous packing material) and tubing. These dressings have all of the disadvantages of non-bioabsorbable dressings as described above. Another disadvantage of these dressing kits is that it is incumbent on the clinician to cut the pad to the correct shape and profile, place the tubing into the pad, and cut the film to the correct size to seal the pad and wound from the atmosphere. The clinician must mate the dressing to the wound and assure that the dressing is sealed from the atmosphere. The job is tedious and requires a great deal of cutting and customization of the pad and film.

Replacement of the above-described wound dressings must be performed with great care. It is still quite common for granulation tissue to become imbedded into the porous wound packing foam even though the pores are quite small. When this occurs, removal of the porous packing must be done very carefully to avoid injuring the new tissue growth and thereby result in bleeding from the wound bed. Blood is an excellent culture medium and bleeding due to dressing removal increases the risk of infection and delays the healing process. In regard to these existing wound treatment kits, it is sometimes impossible to remove the non-bioabsorbable foam without injuring the healing tissue.

Hence, those skilled in the art have recognized a need for a system and method that provide a negative pressure wound dressing that solves the problem of ingrowth of granulation tissue. There has also been recognized by those skilled in the art a need for a negative pressure dressing that is made of bio-absorbable materials and therefore does not need to be removed should ingrowth of granulation tissue occur, and that has minimal risk to the patient when pieces of the dressing are left in the wound for long periods of time. There has also been recognized a need for a negative pressure wound dressing that may be applied by a clinician with minimal experience and training. Still another recognized need is a negative pressure wound dressing that comes in a kit and easily conforms to the wound. The present invention fulfills these needs and others.

### SUMMARY OF THE INVENTION

Briefly and in general terms, the present invention provides a new and improved negative pressure wound dressing system and method for treating a wound with negative pressure. The system includes at least one bioabsorbable component that permits the ingrowth of granulation tissue into the dressing system, thereby making the dressing treatment safer, more efficient, and less painful.

A negative pressure wound dressing system comprises a bioabsorbable wound bed layer, an atmospheric barrier layer disposed over the bioabsorbable wound bed layer, a seal connecting the atmospheric barrier layer with skin surrounding the wound to seal the wound from atmospheric pressure, and a negative pressure generating system having a device located within the wound under the atmospheric barrier layer to apply negative pressure to the wound. In more detailed aspects, the negative pressure generating system comprises a tube having a distal end located within the wound under the atmospheric barrier layer and a proximal end connected to a suction source. Further, the seal comprises an adhesive film layer. Yet in a further aspect, the negative pressure wound dressing system further comprises a fluid communicating layer disposed between the wound bed layer and the atmospheric barrier layer, wherein the fluid communicating layer is formed of a porous material conformable to the shape of the wound and through which fluids produced by the wound may pass. In a more detailed aspect, the fluid communicating layer comprises a bioabsorbable sponge.

In other more detailed aspects, the negative pressure wound dressing system further comprises a breathable layer disposed between the bioabsorbable wound bed layer and the fluid communicating layer through which fluids produced by the wound may pass. The breathable layer comprises multiple perforations through which fluids produced by the wound may pass. Further, the negative pressure generating system comprises a tube having a distal end located within the wound under the atmospheric barrier layer and a proximal end connected to a suction source, and the tube penetrates through an aperture formed in the atmospheric barrier layer and the distal end of the tube connects with the fluid communicating layer whereby fluids produced by the wound that reside in the fluid communication layer can be removed by the tube.

In other aspects in accordance with the invention, the negative pressure wound dressing system comprises a fluid removal system coupled to the tube that removes liquid and debris conducted through the tube from the wound. In a more detailed aspect, the fluid removal system comprises a trap disposed in the communication with the tube to trap liquid and debris conducted through the tube from the wound.

In yet further aspects, at least a portion of the wound bed layer is bonded to the fluid communication layer. The seal has an inner opening that is smaller than the outer size of the atmospheric barrier layer and the seal further has an outer size that is larger than the outer size of the atmospheric barrier layer, wherein the seal overlaps both the atmospheric barrier layer and skin at the wound edges. The seal comprises adhesive disposed at portions of the seal that contact the atmospheric barrier layer and the skin at the wound edges, whereby when mounted to the atmospheric barrier layer and the skin at the wound edges, the seal seals the would from atmospheric pressure. In a much more detailed aspect, the seal is configured as a solid sheet having a size that entirely covers the atmospheric barrier layer and skin at the wound edges. However in another aspect, the seal is configured as a frame with an opening wherein the seal covers only outer edges of the atmospheric barrier layer and skin at the wound edges. And in yet even more detailed aspects, the frame seal is formed from a sheet of seal material within which multiple individual frame seals have been at least partially formed and each of which may be used on the wound by separating the desired frame seal from the sheet. Additionally, the seal is formed from a sheet of seal material within which multiple individual seals have been at least partially formed and each of which may be used by separating the desired seal from the sheet.

In another aspect, the atmospheric barrier layer is formed from a sheet of atmospheric barrier layer material within which multiple individual atmospheric barriers have been at least partially formed and each of which may be used by separating the desired atmospheric barrier from the sheet. Further, a bacterial growth inhibitor is formed as part of at least one of the wound bed layer and the fluid communicating layer.

In kit aspects in accordance with the invention, there is provided a negative pressure wound dressing kit that comprises a bioabsorbable wound bed layer, a fluid communicating layer, an atmospheric barrier layer, a tube including a generally central channel, and an adhesive film layer. In a further aspect, the fluid communicating layer is formed of a bioabsorbable material. In another aspect, the negative pressure wound dressing kit of claim 20 further includes a breathable layer of silicon.

In other aspects, at least the bioabsorbable fluid communicating layer, the tube, and the atmospheric barrier layer are connected together during pre-assembly. In a different aspect, at least the tube and the atmospheric barrier layer are connected together during pre-assembly. In another different aspect, at least the fluid communicating layer and the tube are connected together during pre-assembly. And in yet a further aspect, at least the bioabsorbable wound bed layer, and the breathable layer of silicon are bonded together during pre-assembly.

Turning now to further more detailed aspects in accordance with the invention, the negative pressure wound dressing kit further comprises a supply of adhesive. Further, a dressing kit comprises a plurality of different size atmospheric barrier layers and a plurality of adhesive film layer frames arranged in concentric fashion and separated by perforations, wherein each frame has a central inner opening smaller in dimension than a corresponding atmospheric barrier layer and an outer size larger in dimension than the outer size of a corresponding atmospheric barrier layer. In yet another kit aspect, a bacterial growth inhibitor is part of at least one of the wound bed layer and the fluid communicating layer.

Turning now to a method in accordance with the invention, there is provided a method of treating a wound having a wound bed with a negative pressure dressing that comprises disposing a bioabsorbable wound bed layer into the wound bed, covering the bioabsorbable wound bed layer with an atmospheric barrier layer, sealing the atmospheric barrier layer to seal the wound from atmospheric pressure, disposing a negative pressure device in the wound under the atmospheric barrier layer, and applying negative pressure to the wound through the negative pressure device to lower the pressure within the wound to a level that is less than atmospheric pressure. In another aspect, the method further comprises the step of removing fluids produced by the wound from the wound bed. In yet a further aspect, the method comprises the step of disposing a fluid communicating layer that is porous so that fluids produced by the wound may pass through it between the wound bed layer and the atmospheric barrier layer. In further detail, the step of disposing a fluid communicating layer comprises disposing a bioabsorbable communicating layer that is porous so that fluids produced by the wound may pass through it between the wound bed layer and the atmospheric barrier layer.

In other more detailed aspects of the invention, the steps of disposing a negative pressure device in the wound under the atmospheric barrier layer and applying negative pressure to the wound through the negative pressure device comprise inserting a distal end of a tube into the fluid communicating layer and connecting the proximal end of the tube with a suction source; and applying suction to the proximal end of the tube to thereby lower the pressure below atmospheric pressure in the wound bed. In yet further detail, the method further includes the step of disposing a breathable layer of silicone between the wound bed layer and the fluid communicating layer. And yet further, the steps of disposing a bioabsorbable wound bed layer and the fluid communicating layer further include disposing a bacterial growth inhibitor in the wound that is part of at least one of the wound bed layer and the fluid communicating layer.

Other features and advantages of the invention will become more apparent from the following detailed description of preferred embodiments of the invention, when taken in conjunction with the accompanying exemplary drawings.

### BRIEF DESCRIPTION OF THE DRAWINGS

FIG. 1 is an exploded view of a negative pressure wound dressing system in accordance with aspects of the invention showing four layers, at least one of which comprises a bioabsorbable material, and showing a tube with which the pressure inside the dressing is reduced below atmospheric pressure;

FIG. 2 is a cross sectional view through a dressed wound bed illustrating the application of the dressing system shown in FIG. 1 to seal the wound bed from atmospheric pressure and showing the tube shown in FIG. 1 being connected to a suction source to lower the pressure within the wound bed;

FIG. 3 is an embodiment of a fluid communication layer having a hemispheric top surface shape;

FIG. 4 shows an adhesive film layer sheet having perforations that may be used to separate the film layer sheet into multiple concentric adhesive film layer frames having different sizes from which a frame having the desired size can be selected for use on the patient's particular wound by the clinician;

FIG. 5 shows an exploded view of multiple concentric adhesive film layer frames formed from the same adhesive film layer sheet of FIG. 4 that was perforated for easy separation of the individual frames;

FIG. 6 shows a figurative exploded view of three possible sizes of an atmospheric barrier layer, all of which can be obtained from a single atmospheric barrier layer sheet having perforations defining each atmospheric barrier, with the particular desired size of the atmospheric barrier layer desired by the clinician obtained by merely separating an outer frame at the perforations of the larger sheet to leave the smaller desired size, or by using the large sheet unaltered;

FIG. 7 shows a top view of a larger size atmospheric barrier layer having stamped perforations to enable a clinician to either use the entire larger sized sheet on a wound or to separate the layer at a perforation by removing an outer frame leaving a smaller sized barrier layer to be used on a wound as was shown in FIG. 6;

FIG. 8 is a perspective view illustrating the mounting of a frame of adhesive film sized to fit over a corresponding pre-sized sheet of atmospheric barrier layer and indicating the respective overlap to result in the wound being sealed from atmospheric pressure;

FIG. 9 is a top view illustrating the frame of adhesive film overlaid upon the corresponding pre-sized sheet of atmospheric barrier layer as shown in FIG. 8;

FIG. 10 shows a cross section through an embodiment of a wound dressing in accordance with aspects of the invention with a breathable layer of silicone disposed between the wound bed layer and the fluid communicating layer; the ability of the silicone layer to “breathe” thus allows fluids produced by the wound to pass through the breathable layer to the fluid communicating layer for removal by the suction system;

FIG. 11 shows an example of a breathable layer of silicone of FIG. 10 including the multitude perforations through the layer resulting in the ability to breathe and conduct fluids produced by the wound for removal; and

FIG. 12 illustrates the contents of a negative pressure wound dressing kit in accordance with aspects of the invention.

### DETAILED DESCRIPTION OF PREFERRED EMBODIMENTS

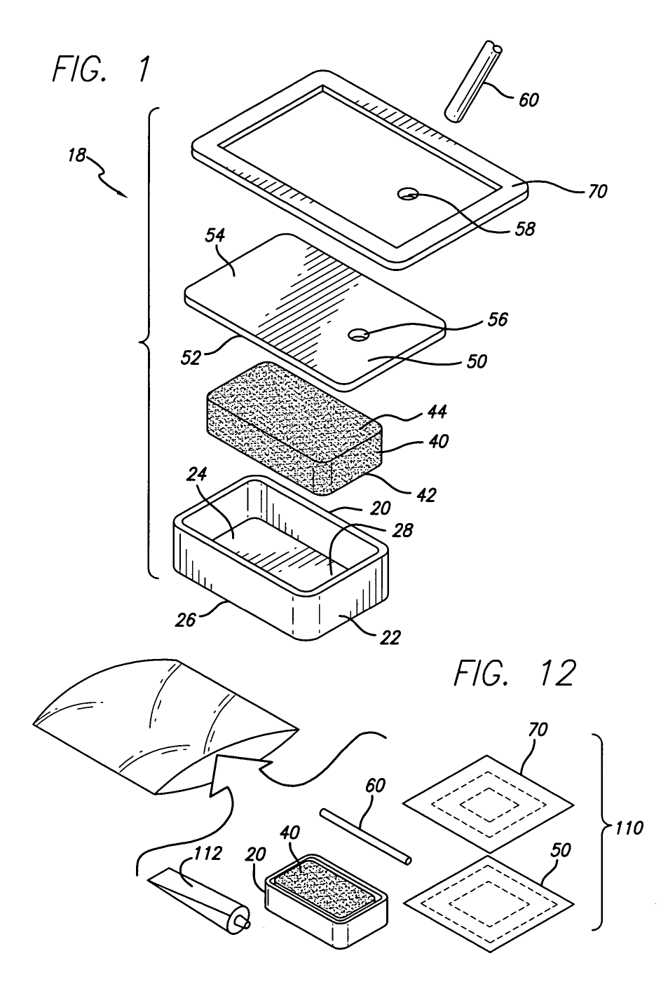

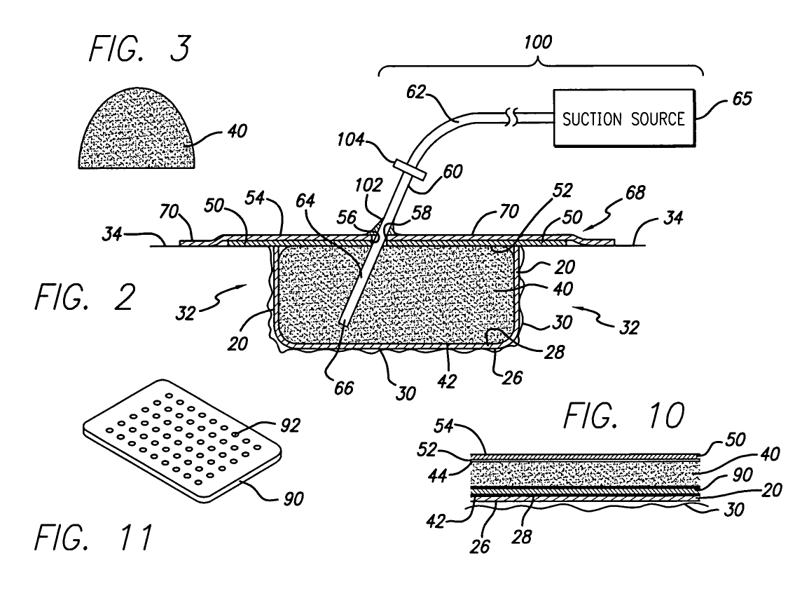

Referring now to the drawings in more detail, which are not intended to be limiting but instead are provided for purposes of illustration and by way of example, and in which like reference numerals are used to refer to like or corresponding elements in the different figures of the drawings, FIGS. 1 and 2 show an embodiment of a laminar construction negative pressure wound dressing in accordance with aspects of the invention. As illustrated, the negative pressure wound dressing system 18 includes a bioabsorbable wound bed layer 20 having side walls 22 and a bottom wall 24. The wound bed layer is meant to be applied directly into contact with the wound bed 30 of a patient 32, as shown in FIG. 2. The wound bed layer operates to protect the wound bed of the patient from external contaminants and irritants, so as much contact with the wound as possible is preferable. Accordingly, the wound bed layer may be quite thin and may have a shape that somewhat resembles a wound shape. In the case of FIG. 1, the sides and bottom are shown but as a practical matter, the layer may simply be a very thin flat sheet. The wound bed layer has a first side 26 facing the wound bed and a second side 28 facing outward from the wound.

The bioabsorbable wound bed layer 20 accommodates and even encourages ingrowth of granulation tissue. This is because the wound bed 30 is partially porous, and fibroblasts formed by the body during healing and in reaction to the presence of the wound bed layer can grow into and enter the wound bed layer. Additionally, its porosity permits fluids produced by the wound to pass through it. In accordance with an aspect of the invention, the bioabsorbable wound bed layer is made of a material that is absorbable by the body. In at least one embodiment, the bioabsorbable wound bed layer is made of an absorbable collagen layer. Such materials that are manufactured from collagen offer complete compatibility and bioabsorption by the human body. Materials made by Integra LifeSciences Corporation, Plainsboro, N.J., such as “Integra Dermal Regeneration Template” are representative of this type of product. In other embodiments, other bioabsorbable materials, naturally occurring or man made, well known in the art may be used in the manufacture of the bioabsorbable wound bed layer.

The bioabsorbable wound bed layer 20 provides a matrix or structural framework for the ingrowth of healing tissue. In other words, the patient's own tissue may grow into the structural framework provided by the bioabsorbable wound bed layer, thereby slowly incorporating and/or replacing the bioabsorbable wound bed layer with the patient's own tissue. Because the wound bed layer is bioabsorbable, it does not need to be removed during dressing changes, and ingrowth of granulation tissue does not need to be inhibited or prevented. In fact, growth of granulation tissue into the wound bed layer may be encouraged to speed wound healing. During dressing changes, the old bioabsorbable wound bed layer may be left behind and a new bioabsorbable wound bed layer placed upon the old layer. Because the wound bed layer is not removed, the chance of tearing tissue and causing bleeding and possible consequential infection are avoided. As the bioabsorbable wound bed layers are added over time, the wound 30 will close and heal over with epithelial tissue. In one embodiment, the thickness of the wound bed layer is selected so that the normal tissue growth rate that occurs between dressing changes (one to five days) is accommodated by the bioabsorbable wound bed layer. For example, a thickness of the wound bed layer of 0.5 to 5 mm is appropriate for most patients under normal circumstances.

With continuing reference to FIGS. 1 and 2, a second layer is termed a fluid communicating layer 40 and is formed of a porous sponge material that is conformable to the size and depth of the wound 30. In at least one embodiment, the fluid communicating layer may be made from a non-absorbable foam. The fluid communicating layer has a first side 42 that is disposed adjacent the bioabsorbable wound bed layer 20 and a second side 44 facing outward from the wound. The pores of the fluid communicating layer are not limited to any particular size. This is because ingrowth of granulation tissue into the fluid communicating layer is limited by the presence of the bioabsorbable wound bed layer. However, the fluid communicating layer, in at least one preferred embodiment, is a bioabsorbable sponge made of a porous collagen that is manufactured to include fluid communication pathways. These materials are commercially available and are of the type manufactured by Integra LifeSciences, Plainsboro, N.J., called Helitene® and Helistat®. Helistat® is an absorbable collagen hemostatic sponge, and Helitene® is an absorbable collagen hemostatic agent in fibrillar form. The porosity of the fluid communication layer permits entry into it of fluids produced by the wound. As discussed above, such fluids are removed to aid in wound healing.

The advantage of using a bioabsorbable material for the fluid communicating layer 40 is that pieces of bioabsorbable sponge can be left behind in the wound 30 without causing a severe tissue reaction or infection. The non-absorbable wound filling sponge presently commercially available is made of an open cell polyurethane or polyether foam. The manufacturer cautions in its instructions that this non-absorbable sponge must be cut at a distance from the open wound. As mentioned above in the Background section, the pieces of non-absorbable sponge placed in the wound must also be counted and recorded by the clinicians. This is because pieces of non-absorbable polyurethane or polyether foam left in the wound for more than five days will almost certainly cause severe tissue reactions, including inflammation and infection. Inflammation and infection will delay or completely prevent wound healing. In comparison, pieces of bioabsorbable material used in accordance with an embodiment of the present invention, if left in the wound, are simply absorbed by the body, markedly decreasing the risk of tissue reaction and infection. Furthermore, bioabsorbable materials can be safely cut directly over the wound, enhancing the ability of the clinician to fit the bioabsorbable sponge to the patient's wound contours. Furthermore, bioabsorbable materials are more user friendly for an inexperienced clinician, who using the older non-absorbable dressings could have inadvertently left non-absorbable contaminants remaining in the wound.

The fluid communicating layer 40 sponge should preferably be between one and one-hundred millimeters (“mm”) thick and able to conform to various wound 30 depths and shapes. The fluid communicating layer however can be provided in various sizes and shapes, for example, cubes or spheres, or hemispheres. Furthermore, more than one sponge can be included in the fluid communicating layer if necessary for a single very large wound. Since pore size in the fluid communicating layer is of little or no concern in accordance with aspects of the invention, that is, ingrowth of granulation tissue into the fluid communicating layer is of little concern due to the existence of the bioabsorbable wound bed layer, the orientation of the fluid communicating layer to the wound bed is not important. The clinician therefore can cut pieces of bioabsorbable sponge and place them in the wound bed layer without regard to which side of the sponge must be adjacent the wound bed layer.

In at least one embodiment, the fluid communicating layer 40 is in the shape of an inverted hemisphere as shown in FIG. 3. Other effective shapes will be readily recognized by those skilled in the art. The fluid communicating layer can be cut to conform to the wound shape 30, so that the fluid communicating layer is level with the top of the wound or skin surface. A fluid communicating layer that is level with the top of the wound may be easier to seal with an atmospheric barrier layer (discussed below) and will also be more comfortable for the patient to lie upon. As negative pressure is applied to the wound dressing, the dressing will tend to collapse into the wound. Because of this, some clinicians may prefer to make the dressing somewhat larger than the wound so that after it collapses due to the application of negative pressure, the dressing will be level with the surrounding skin. Furthermore, in at least one embodiment, the wound bed layer 20 and/or the fluid communicating layer can include a bacterial growth inhibitor, for example an antibiotic.

As used herein, “negative pressure” is meant to describe a pressure that is less than atmospheric pressure, which is 760 mm Hg at sea level. A “negative pressure” environment is an environment in which the pressure within the environment is less than the room pressure of the air surrounding the environment. For example, suction applied to an atmospherically sealed environment, such as a sealed wound bed, will result in a pressure within that wound bed that is lower than the room pressure surrounding the patient and that wound bed pressure may be referred to herein as “negative pressure.” A lower pressure in an atmospherically sealed environment, such as a sealed wound bed, may also be referred to herein as a “vacuum” or “partial vacuum.”

In at least one embodiment, the bioabsorbable fluid communicating layer 40 is bonded by means of a medical grade adhesive to the bioabsorbable wound bed layer 20. The bonding is preferably accomplished in a way that does not affect fluid communication between the two layers. In at least one embodiment, the bonding as applied in an interrupted “spot welding” manner to allow fluid communication around and between the bonded areas. Medical grade adhesives that are available for this type of bonding are well known in the art. In at least one embodiment, the bonding is be done by the manufacturer before shipping to the clinician. Pre-bonding makes it easier for an inexperienced clinician to apply the dressing.

In at least one embodiment, no adhesive is disposed upon either the fluid communicating layer 40 or the bioabsorbable wound bed layer 20. Instead, the fluid communicating layer and the bioabsorbable wound bed layer are provided as separate components. The dressing is applied without bonding these two layers together, thereby making it very easy to remove the fluid communicating layer at the subsequent dressing change, and leaving the bioabsorbable wound bed layer behind. Alternatively, the fluid communicating layer and the wound bed layer components can then be bonded together by the clinician at the bedside. For example, the clinician can dispense a medical bonding adhesive from a tube or a syringe. Bonding at the bedside by the clinician allows for the individual trimming of the fluid communicating layer and the absorbable wound bed layer before they are bonded to each other. Individually trimming allows the clinician to create sizes and shapes that best conform to the area and depth of the individual patient's wound. This may be particularly useful for an experienced clinician.

A further layer comprises an atmospheric barrier layer 50 placed upon the fluid communicating layer 40. The atmospheric barrier layer has a first side 52 facing the wound 30 and a second side 54 facing outward from the wound. The atmospheric barrier layer should be large enough in surface area to cover the entire wound opening and overlap the skin 34 by a broad enough margin to allow an atmospheric seal to be established with the skin. The atmospheric barrier layer is preferably large enough to completely overlap the fluid communicating layer and cover at least one-half inch (1.3 cm) of the skin surrounding the wound edges. The atmospheric barrier layer must be at least impermeable enough to the passage of atmospheric air to allow standard hospital suction devices applied to the tube 60 shown in FIG. 1 and FIG. 2 to remove air at a faster rate than air can enter the wound through the atmospheric barrier layer. In at least one preferred embodiment, the atmospheric barrier layer is generally impermeable to air. Preferably, the atmospheric barrier layer also is impermeable to bacteria, dust, and moisture. A bacteria impermeable atmospheric barrier layer prevents contamination of the wound between dressing changes by preventing bacteria entry to the wound bed. The atmospheric barrier layer should preferably be made from a material known in the art to cause minimal skin irritation.

In some embodiments the atmospheric barrier layer 50 may be impermeable to gases. In yet other embodiments, the atmospheric barrier layer may be partially permeable to gases. In some embodiments the atmospheric barrier layer may be impermeable to moisture. In yet other embodiments, the atmospheric barrier layer may be partially permeable to moisture. In some embodiments, the atmospheric barrier layer can be made of, for example, a plastic or polyurethane film. In other embodiments, the atmospheric barrier layer can be made from a silicone or rubber material. Other appropriate materials having the above characteristics are well known in the art and need not be described in more detail herein. In at least one embodiment, the first side 52 of the atmospheric barrier layer is bonded to the second side 44 of the fluid communicating layer by the manufacturer.

An atmospheric seal 68 that substantially excludes gas and moisture is provided between the atmospheric barrier layer 50 and the surrounding skin 34. The atmospheric seal must be impervious enough to maintain a negative pressure environment in the wound bed 30 without any significant leakage of atmospheric air into the wound. The atmospheric seal maintains a pressure differential across the atmospheric barrier layer 50, between the outside atmospheric pressure and the inside (wound-side) negative pressure. The atmospheric seal in some embodiments may be provided by the atmospheric barrier layer and the surrounding skin 34 by applying an adhesive, for example Stomahesive® paste, to the atmospheric barrier layer and sticking the atmospheric barrier layer to the surrounding and underlying skin. In other embodiments, the atmospheric seal 68 may be provided by an adhesive film layer 70 that overlaps both the atmospheric barrier layer 50 and the skin 34 that is adjacent the wound 30 edges.

In at least one embodiment, the atmospheric seal 68 is provided by an adhesive that is pre-applied by the manufacturer at least around the outer one-half to two inch (1.3 to 5 cm) perimeter of the first side 52 of the atmospheric barrier layer 50. The generally central area of the atmospheric barrier layer may also have adhesive applied to it, or in some embodiments, the generally central area of the atmospheric barrier layer may be free of adhesive. In one embodiment, a non-stick backing is provided that covers the adhesive surface during storage. The non-stick backing is removed by the clinician at the time the perimeter of the atmospheric barrier layer is applied to the patient's skin 34. The pre-applied adhesive adheres to the skin surrounding the wound 30 when the dressing 18 is positioned in the wound 30, thereby providing the seal. The atmospheric barrier layer can be manufactured in various sizes and shapes, for example square, round, or elliptical, to accommodate various patients' wounds.

In yet some other embodiments, in addition to or as an alternative to the adhesive being directly applied to the first side 52 of the atmospheric barrier layer 50, an adhesive film layer 70 (such as those commercially available as Opsite® or Tegaderm®) is provided. The adhesive film layer connects with the atmospheric barrier layer and the skin 34, thereby providing an atmospheric seal 68 for the wound 30. The atmospheric seal maintains a pressure differential, with negative pressures on one side of the atmospheric barrier layer and atmospheric room pressure on the other side of the atmospheric barrier layer. The adhesive film layer should keep substantially all moisture and gas from entering around the atmospheric barrier layer into the wound cavity. In a preferred embodiment, at least one of the sheets of the adhesive film layer 70 is supplied with a pre-applied adhesive and a removable non-stick backing. The non-stick backing is provided that covers the adhesive surface of the adhesive film layer during storage. The non-stick backing is removed by the clinician from the adhesive film layer just prior to application on the patient.

In at least one embodiment, a pre-sized sheet of atmospheric barrier layer 50 is provided along with its complementary pre-sized sheet or frame of pre-cut adhesive film layer 70. Several sizes of these complementary pairs of atmospheric barrier layer and corresponding adhesive film layer can be provided in a kit. This saves the clinician addition time and effort in cutting, applying, and changing the dressing.

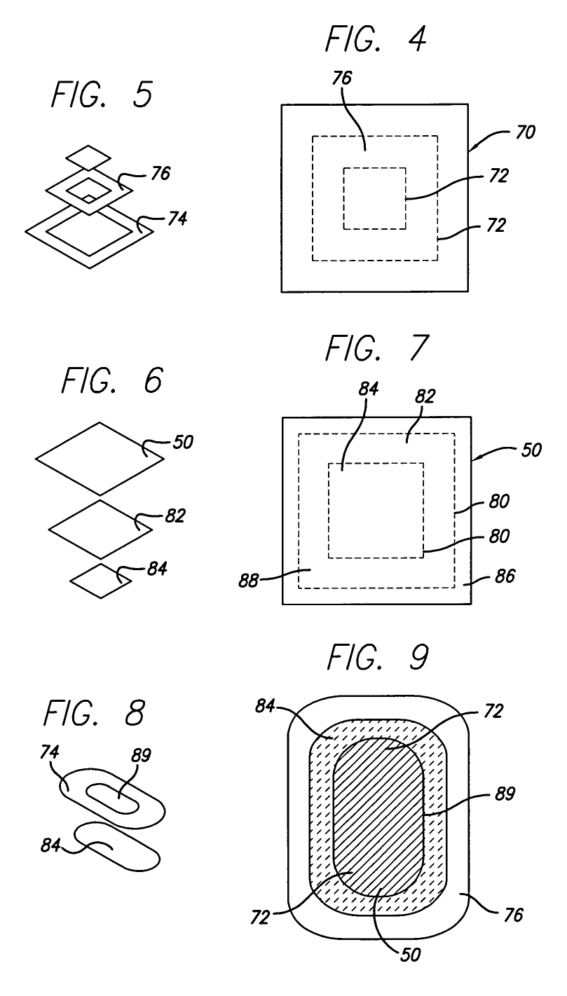

Referring now to FIG. 4 and FIG. 5, in one embodiment a perforated sheet of adhesive film layer 70 is provided. The adhesive film layer 70 is manufactured as a sheet with perforations 72 formed in the sheet by stamping or other means to result in multiple adhesive film frames 74 and 76 as shown in the exploded view of FIG. 5. They may take the form of various shapes, such as annular rings, ellipses, or rectangles and because of the perforations; they are easily separable from each other. The perforations of the adhesive film layer are placed in predetermined locations to provide frame sizes that overlap predetermined sizes of sheets of atmospheric barrier layers 50. The adhesive film layer 70 is sized large enough to also overlap the skin 34 of the patient when applied to the atmospheric barrier layer sheet, thereby sealing the atmospheric barrier layer 50 to the patient's skin surface, see FIG. 1. The adhesive film layer 70 preferably has a non-stick backing that is removed by the clinician before adhering the adhesive film layer 70 to the patient.

The stamped or perforated sheet of adhesive film layer however does not need to be utilized in just picture frame shapes. In the embodiment described above, a frame of adhesive film layer 70 may be used to cover only the outer periphery of an atmospheric barrier layer sheet 50. Alternatively however, one or more of the outer frames of adhesive film layer may be removed from the large sheet and discarded. This will leave remaining a smaller solid sheet of adhesive film layer 70. The solid sheet of adhesive film layer 70, without a central opening as a frame has, can then be used to cover the entire outer surface of an atmospheric barrier layer sheet 50.

A similar approach can be used with the atmospheric barrier layer 50 to result in multiple sized layers from a single layer sheet. In the embodiment shown in FIGS. 6 and 7, there is a large sheet of an atmospheric barrier layer 50 that is concentrically stamped or perforated 80 during manufacture. The perforations permit the larger sheet of atmospheric barrier layer 50 to be separated into pre-determined smaller size sheets 82 and 84 by the clinician when needed for use. Alternatively, the entire large sheet 50 may be used as one piece. Upon dressing the wound 30 and determining the size of the atmospheric barrier layer needed, the clinician may use the entire sheet 50 or may separate the sheet into the next smaller size 82 by separating it at the perforations 80 from the outer border 86. Similarly, if the smallest size sheet 84 is desired for use, the clinician may simply separate it from the outer border 86 plus 88 by separating them at the appropriate perforations. Thus a single atmospheric barrier layer sheet 50 actually furnishes the clinician the choice an atmospheric barrier layer of three sizes.

Referring now also to FIG. 8 and FIG. 9, it is illustrated how a frame of adhesive film layer overlaps a corresponding size sheet of atmospheric barrier layer. The adhesive film layer and atmospheric barrier layer are elliptically shaped in these figures. In this case, the atmospheric barrier layer 84 is similar to the smaller size layer of FIG. 7 (84) and has been formed by separating it at the appropriate perforations 80 from the outer borders 86 and 88. The adhesive film layer 70 has been formed into a frame 76 similar to the mid size of FIG. 4 by separation at appropriate perforations. The adhesive film layer 70 is a frame in shape and has a generally central inner opening 89 smaller in size than the outer size of the corresponding sheet of atmospheric barrier layer 84. The adhesive film layer further has an outer size that is larger than the outer size of the corresponding sheet of atmospheric barrier layer 84, wherein the adhesive film layer is capable of overlapping both the atmospheric barrier layer and the skin of the patient, thereby adhering to the atmospheric barrier layer and the skin, and forming an atmospheric seal when applied.

Alternative shapes other than circles, ellipses, or rectangles for the atmospheric barrier layer 50 and the adhesive film layer 70 are possible. The precut sheets of frame shaped adhesive film layer allow the clinician to select the best fitting inner and outer circumferences for the frame of adhesive film layer, as required by the patient's specific wound 30 size and shape and skin sealing requirements. The skin 34 is preferably cleaned and prepared prior to placing the adhesive film layer.

It is advantageous for the adhesive film layers to simply be peeled off a sheet and applied to the patient 32 without the clinician taking the individual time and effort to cut out a pattern into the adhesive film layer. Other ways of supplying concentric frames of adhesive film layer should be apparent to those skilled in the art in view of the embodiments discussed and shown herein.

A negative pressure generating system 100 provides negative pressure to the wound bed 30. Referring back again to FIGS. 1 and 2, the negative pressure generating system comprises a tube 60 connected to a source of suction 65. The tube 60 has a distal end 64 and a proximal end 62 with a generally central axial channel 66 connecting the two. The distal end 64 of the tube 60 inserts into the fluid communicating layer 40. The proximal end 62 of the tube 60 is connected to a source of suction 65; thereby providing negative pressure to the wound dressing 18. The source of suction may be any negative pressure generating device known in the art, for example, hospital wall suction, a suction pump, a portable suction pump, or an expandable canister. A portable canister is particularly useful during patient transportation to maintain a negative pressure in the wound bed for short periods of time while the patient is undergoing treatments, therapy, or diagnostic testing, for example.

The tube 60 may be made of any suitable material, for example, rubber, silicone, or plastic. The tube should be rigid enough to avoid collapse of the tube wall when negative pressure is applied through the channel 66 and also should resist pinching off the suction force with movement of the patient 32. In at least one embodiment, the distal end 64 of the tube in the preferred embodiment penetrates the atmospheric barrier layer 50 through an aperture 56. The tube may also penetrate the adhesive film layer 70 through an aperture 58 in the case where the adhesive file layer covers the aperture of the atmospheric barrier layer. In at least one embodiment, the tube is sealed to the atmospheric barrier layer aperture by the manufacturer, thereby eliminating the need for the clinician to bond and seal the tube to the atmospheric barrier layer. In yet another embodiment, the tube is sealed to the atmospheric barrier layer by the clinician, for example with Stomahesive® adhesive 102 or waterproof tape. The tube may likewise be sealed to the adhesive film layer aperture by adhesive or tape. The distal end 64 of the tube may either be inserted into the fluid communicating layer by the clinician or pre-inserted into the fluid communicating layer at the factory.

The tube 60 has a generally central axial channel 66 for flow of liquids and debris out of the wound 30 towards the source of suction 65. Preferably, a trap 104 is placed somewhere between the dressing 18 and the suction source, such as within the tube line in order to catch the liquids and debris expressed from the wound. Traps are well known to those skilled in the art. They come in multiple forms, and hence, no further detail is provided here. The distal end 64 of the tube in some embodiments has multiple side wall perforations (not shown) that provide additional places for communication and distribution of the negative pressure generating suction force within the fluid communicating layer 40. If there are additional side wall perforations in the tube, then care should be taken to assure that the side wall perforations are contained within the fluid communicating layer so that negative pressure to the wound bed is not diminished. Locating perforations outside of the atmospheric barrier layer 50 should be avoided so that negative pressure to the wound bed is not lost.

Additional negative pressure generating devices may be used with the various embodiments. For example, a manual or mechanical suction pump 65 may be attached directly to the aperture 56 in the atmospheric barrier layer 50. A mechanical pump may be driven by a wall outlet source of electrical power or a portable power source, for example a battery. An expandable canister may also be used as the suction source. A suction pump with a suction cup or gasket may be attached, and preferably sealed in place, over the aperture 56 in the atmospheric barrier layer. If an adhesive film layer 70 with an aperture 58 is applied, the suction cup or gasket should be positioned to fit over at least the aperture 58 in the adhesive film layer 70. Furthermore, the atmospheric barrier layer need not be limited in configuration to a flat sheet. The atmospheric barrier layer, for example, may be dome-shaped or bell-shaped with a periphery sized to overlap the wound edges to be treated.

As shown in FIG. 10 and FIG. 11, in at least one embodiment, an additional removable breathable layer of silicone 90 may be disposed between the first side 42 of the fluid communicating layer 40 and the wound bed layer 20. The breathable layer of silicone includes multitude perforations 92 that permit fluid communication between the wound bed layer 20 and the fluid communicating layer. Fluids produced by the wound will pass through the breathable layer to the fluid communication layer for remove. The breathable layer of silicone is bonded to the first side 42 of the fluid communicating layer in at least one embodiment. The bonding is distributed in small spots, taking care to leave adequate areas without adhesive bond so that fluid may easily flow through the breathable silicone layer to the fluid communicating layer. In yet other embodiments, an empty space is left between the breathable silicone layer and the fluid communicating layer. The breathable layer of silicone is advantageous in further preventing the migration of granulation tissue into the adjacent sponge-like fluid communicating layer.

Materials included in the dressing may further incorporate antimicrobial or anti-infective agents to minimize infection of the wound site. Anti-infectives, for example, silver ion solutions, or active antibiotics such as rifampin or vancomycin, may be included in the dressing 18.

The invention further includes a pre-assembled product including the bioabsorbable wound bed layer 20 connected to the first side 42 of the fluid communicating layer 40. The second side 44 of the fluid communicating layer is connected to the first side 52 of the atmospheric barrier layer 50. The distal end 64 of the tube 60 is inserted through the atmospheric barrier layer 50 and into the fluid communicating layer 40. Yet another embodiment is the pre-assembled product, above, further including the breathable layer of silicone 90 connected between the wound bed layer 20 and the first side of the fluid communicating layer. The advantage of pre-assembling the layers and the tube of the negative pressure wound dressing system is that it saves the clinician time and effort, and assures that even the less experienced technicians can properly place and change the wound dressing.

Still another aspect of the present invention is a method of treating a chronic wound with a negative pressure dressing. The method includes disposing a bioabsorbable wound bed layer 20 on a wound bed 30; sealing an atmospheric barrier layer 50 to the skin 34 surrounding the wound; and placing the wound under negative pressure using a negative pressure generating device.

In yet another embodiment, the method includes the steps of disposing a bioabsorbable wound bed layer 20 on the first side 42 of a bioabsorbable fluid communicating layer 40, applying an atmospheric barrier layer 50 over the second side 44 of the bioabsorbable fluid communicating layer, inserting a tube 60 through the atmospheric barrier layer into the bioabsorbable fluid communicating layer, sealing the entry point aperture 56 of the tube through the atmospheric barrier layer, and applying an adhesive layer 70 over the atmospheric barrier layer and skin 34, thereby providing an atmospheric seal over the wound and the fluid communicating layer, connecting a suction source 65 to the tube to create a negative pressure on the wound side of the atmospheric barrier. In at least one embodiment the method further includes placing a suction trap 104 between the dressing 18 and the suction source 65 to trap fluids removed from the wound 30.

Yet another method of treating a chronic wound 30 with a negative pressure dressing 18 in accordance with aspects of the invention includes disposing a bioabsorbable wound bed layer 20 on a breathable layer of silicone 90, bonding the breathable layer of silicone to a bioabsorbable fluid communicating layer 40, applying an atmospheric barrier layer 50 over another side of the bioabsorbable fluid communicating layer, inserting a tube 60 into the bioabsorbable fluid communicating layer, sealing the tube to the atmospheric barrier layer, and applying an adhesive layer 70 over the atmospheric barrier layer and the skin 34, thereby providing an atmospheric seal over the wound and the fluid communicating layer, connecting a suction source 65 to the tube to create a negative pressure on the wound side of the atmospheric barrier. In at least one embodiment the method further includes placing a suction trap 104 between the dressing 18 and the suction source 65 to trap fluids removed from the wound.

Referring now to FIG. 12, there is shown a negative pressure wound dressing kit 110 or assembly comprising a bioabsorbable wound bed layer 20; a fluid communicating layer 40; an atmospheric barrier layer 50; a tube 60 including a generally central channel; and an adhesive film layer 70. In at least one embodiment, the fluid communicating layer is bioabsorbable. In yet another embodiment the kit includes a breathable layer of silicon (see FIG. 11). In still another embodiment, the fluid communicating layer, the tube, and the atmospheric barrier layer are connected together during pre-assembly at the factory. In another embodiment of the kit, at least the tube and the atmospheric barrier layer are connected together during pre-assembly. In yet another embodiment of the kit, the fluid communicating layer and the tube are connected together during pre-assembly. In another embodiment of the kit, the bioabsorbable wound bed layer and the breathable layer of silicon are bonded together during pre-assembly. In yet another embodiment, the kit includes a supply of adhesive 112, such as that sold as Stomahesive® adhesive. In another embodiment, the kit includes a multitude of different size atmospheric barrier layer sheets, each corresponding in size to a matching adhesive film layer frame; and a multitude of adhesive film layer frames arranged in concentric fashion and separated by perforations, wherein each frame has a central inner opening smaller in dimension than the corresponding atmospheric barrier layer sheet, and an outer perimeter larger in dimension than the corresponding atmospheric barrier layer sheet.

Thus there has been provide a new and useful wound dressing comprising bioabsorbable material and a negative pressure system to facilitate wound healing.

The invention may be embodied in other forms without departure from the scope and essential characteristics thereof. The embodiments described therefore are to be considered in all respects as illustrative and not restrictive. Although the present invention has been described in terms of certain preferred embodiments, other embodiments may occur to those skilled in the art that fall within with the scope of the invention. Accordingly, the scope of the invention is intended to be defined only by reference to the appended claims.

We claim:

1. A negative pressure wound dressing system comprising:
a bioabsorbable wound bed layer;
an atmospheric barrier layer disposed over the bioabsorbable wound bed layer;
a seal connecting the atmospheric barrier layer with skin surrounding the wound to seal the wound from atmospheric pressure; and
a negative pressure generating system having a device located within the wound under the atmospheric barrier layer to apply negative pressure to the wound.

2. The negative pressure wound dressing system of claim 1 wherein the negative pressure generating system comprises a tube having a distal end located within the wound under the atmospheric barrier layer and a proximal end connected to a suction source.

3. The negative pressure wound dressing system of claim 1 wherein the seal comprises adhesive located on the atmospheric barrier layer.

4. The negative pressure wound dressing system of claim 1 further comprising a fluid communicating layer disposed between the wound bed layer and the atmospheric barrier layer, wherein the fluid communicating layer is formed of a porous material conformable to the shape of the wound and through which fluids produced by the wound may pass.

5. The negative pressure wound dressing system of claim 4 wherein the fluid communicating layer comprises a bioabsorbable sponge.

6. The negative pressure wound dressing system of claim 4 further comprising a breathable layer disposed between the bioabsorbable wound bed layer and the fluid communicating layer through which fluids produced by the wound may pass.

7. The negative pressure wound dressing system of claim 6 wherein the breathable layer comprises multiple perforations through which fluids produced by the wound may pass.

8. The negative pressure wound dressing system of claim 2 wherein:
the negative pressure generating system comprises a tube having a distal end located within the wound under the atmospheric barrier layer and a proximal end connected to a suction source; and
the tube penetrates through an aperture formed in the atmospheric barrier layer and the distal end of the tube connects with the fluid communicating layer;
whereby fluids produced by the wound that reside in the fluid communication layer can be removed by the tube.

9. The negative pressure wound dressing system of claim 8 further comprising a fluid removal system coupled to the tube that removes liquid and debris conducted through the tube from the wound.

10. The negative pressure wound dressing system of claim 9 wherein the fluid removal system comprises a trap disposed in the communication with the tube to trap liquid and debris conducted through the tube from the wound.

11. The negative pressure wound dressing of claim 4 wherein at least a portion of the wound bed layer is bonded to the fluid communication layer.

12. The negative pressure wound dressing system of claim 1 wherein the seal comprises a film layer having an inner opening that is smaller than the outer size of the atmospheric barrier layer and the seal further has an outer size that is larger than the outer size of the atmospheric barrier layer, wherein the seal overlaps both the atmospheric barrier layer and skin.

13. The negative pressure wound dressing of claim 12 wherein the seal comprises adhesive disposed at portions of the seal that contact the atmospheric barrier layer and the skin, whereby when mounted to the atmospheric barrier layer and the skin, the seal seals the wound from atmospheric pressure.

14. The negative pressure wound dressing of claim 1 wherein the seal is configured as a solid sheet having a size that entirely covers the atmospheric barrier layer and extends to cover skin located about the wound.

15. The negative pressure wound dressing of claim 1 wherein the seal is configured as a frame with an opening wherein the seal covers only outer edges of the atmospheric barrier layer and skin located about the wound.

16. The negative pressure wound dressing of claim 15 wherein the frame seal is formed from a sheet of seal material within which multiple individual frame seals have been at least partially formed and each of which may be used on the wound by separating the desired frame seal from the sheet.

17. The negative pressure wound dressing of claim 1 wherein the seal is formed from a sheet of seal material within which multiple individual seals have been at least partially formed and each of which may be used by separating the desired seal from the sheet.

18. The negative pressure wound dressing of claim 1 wherein the atmospheric barrier layer is formed from a sheet of atmospheric barrier layer material within which multiple individual atmospheric barriers have been at least partially formed and each of which may be used by separating the desired atmospheric barrier from the sheet.

19. The negative pressure wound dressing of claim 4 further comprising a bacterial growth inhibitor that is part of at least one of the wound bed layer and the fluid communicating layer.

20. A negative pressure wound dressing kit comprising:
a bioabsorbable wound bed layer;
a fluid communicating layer;
an atmospheric barrier layer; and
a tube including a generally central channel.

21. The negative pressure wound dressing kit of claim 20 wherein the fluid communicating layer is formed of a bioabsorbable material.

22. The negative pressure wound dressing kit of claim 20 further including a breathable layer of silicon.

23. The negative pressure wound dressing kit of claim 20 wherein at least the bioabsorbable fluid communicating layer, the tube, and the atmospheric barrier layer are connected together during pre-assembly.

24. The negative pressure wound dressing kit of claim 20 wherein at least the tube and the atmospheric barrier layer are connected together during pre-assembly.

25. The negative pressure wound dressing kit of claim 20 wherein at least the fluid communicating layer and the tube are connected together during pre-assembly.

26. The negative pressure wound dressing kit of claim 22 wherein at least the bioabsorbable wound bed layer and the breathable layer of silicon are bonded together during pre-assembly.

27. The negative pressure wound dressing kit of claim 20 further comprising a supply of adhesive.

28. The negative pressure wound dressing kit of claim 20 further comprising:
a plurality of different size atmospheric barrier layers; and
a plurality of adhesive film layer frames arranged in concentric fashion and separated by perforations, wherein each frame has a central inner opening smaller in dimension than a corresponding atmospheric barrier layer and an outer size larger in dimension than the outer size of a corresponding atmospheric barrier layer.

29. The negative pressure wound dressing of claim 20 further comprising a bacterial growth inhibitor that is part of at least one of the wound bed layer and the fluid communicating layer.

30. A method of treating a wound having a wound bed with a negative pressure dressing comprising:
disposing a bioabsorbable wound bed layer into the wound bed;
covering the bioabsorbable wound bed layer with an atmospheric barrier layer;
sealing the atmospheric barrier layer to seal the wound from atmospheric pressure;
disposing a negative pressure device in the wound under the atmospheric barrier layer; and
applying negative pressure to the wound through the negative pressure device to lower the pressure within the wound to a level that is less than atmospheric pressure.

31. The method of treating a wound with a negative pressure dressing of claim 30 further comprising the step of removing fluids produced by the wound from the wound bed.

32. The method of treating a wound with a negative pressure dressing of claim 30 further including the step of disposing a fluid communicating layer that is porous so that fluids produced by the wound may pass through it between the wound bed layer and the atmospheric barrier layer.

33. The method of treating a wound with a negative pressure dressing of claim 32 wherein the step of disposing a fluid communicating layer comprises disposing a bioabsorbable communicating layer that is porous so that fluids produced by the wound may pass through it between the wound bed layer and the atmospheric barrier layer.

34. The method of treating a wound with a negative pressure dressing of claim 30 wherein the steps of disposing a negative pressure device in the wound under the atmospheric barrier layer and applying negative pressure to the wound through the negative pressure device comprise:
inserting a distal end of a tube into the fluid communicating layer and connecting the proximal end of the tube with a suction source; and
applying suction to the proximal end of the tube to thereby lower the pressure below atmospheric pressure in the wound bed.

35. The method of treating a wound with a negative pressure dressing of claim 30 further including the step of disposing a breathable layer of silicone between the wound bed layer and the fluid communicating layer.

36. The method of treating a wound with a negative pressure dressing of claim 32 wherein the steps of disposing a bioabsorbable wound bed layer and the fluid communicating layer further include disposing a bacterial growth inhibitor in the wound that is part of at least one of the wound bed layer and the fluid communicating layer.
